(19) United States
(12) Patent Application Publication
(10) Pub. No.: US 2025/0041519 A1
(43) Pub. Date: Feb. 6, 2025

(54) Drug Delivery Investigation Device

(71) Applicant: Becton, Dickinson and Company, Franklin Lakes, NJ (US)

(72) Inventors: Steve Beguin, Nenzlingen (CH); David James Coleman, Shankill (IE); Danielle Aboud, Dublin (IE); Patrick Le Gal Redon, Seyssinet-Pariset (FR); Ronald J. Pettis, Cary, NC (US); Natasha G. Bolick, Raleigh, NC (US)

(21) Appl. No.: 18/705,172

(22) PCT Filed: Nov. 1, 2022
(86) PCT No.: PCT/US22/48530

§ 371 Date: Apr. 26, 2024

Related U.S. Application Data
(60) Provisional application No. 63/274,296, filed on Nov. 1, 2021.

(51) Int. Cl. A61M 5/172; A61M 5/142; A61M 5/168

(52) U.S. Cl. CPC A61M 5/1723; A61M 5/16854; A61M 2005/14252

(57) ABSTRACT

A drug delivery investigation device includes a needle insertion mechanism having a cannula, a flow path having an inlet configured to be in fluid communication with a fluid source and an outlet, connection tubing extending between the outlet of the flow path and the needle insertion mechanism, with the connection tubing in fluid communication with the cannula and the flow path, a flow sensor positioned between the inlet and the outlet of the flow path, and a pressure sensor positioned between the inlet and the outlet of the flow path.

### CROSS-REFERENCE TO RELATED APPLICATION

This application claims priority to U.S. Provisional Application No. 63/274,296, filed Nov. 1, 2021, which is hereby incorporated by referenced in its entirety.

### BACKGROUND OF THE INVENTION

### Field of the Invention

The present disclosure relates to a drug delivery investigation device to study biomechanical tissue response and injection device performance.

### Description of Related Art

Drug delivery devices, such as wearable injection devices, infusion pumps, automatic injectors, and the like, have the benefit of providing therapy to the patient at a location remote from a clinical facility and/or while being worn discretely under the patient's clothing. A wearable medical device, for example, can be applied to the patient's skin and configured to automatically deliver a dose of a pharmaceutical composition within a predetermined time period after applying the wearable medical device to the patient's skin, such as after a 27 hour delay. After the device delivers the pharmaceutical composition to the patient, the patient may subsequently remove and dispose of the device. Similarly, a non-wearable device, such as an auto-injector, can be used to puncture a patient's skin and automatically deliver a dose of a medication from a reservoir and through a needle. Other non-wearable drug delivery devices, such as an infusion pump, can deliver a dose of medication through a catheter or port.

In certain circumstances, due to the medium in which the liquid is being injected, the flow of fluid leaving the device may be impaired, which can lead to increased pressure in the fluid path of the device. When the pressure rises above a certain threshold, the integrity of the fluid path may be compromised causing a leak within the device and a failure to deliver the full dose of medicament. A fluid leak within the device, which may correspond to a pressure drop within the fluid path of the device, may also cause damage to the device and subsequent system failures as well as potential contamination concerns due to contact between the fluid and the device.

Human subcutaneous tissue is composed of various cell types, extracellular matrix (ECM) constituents, microstructures, and macroscopic arrangement of cells and ECM. Those elements contribute to the mechanical properties of the tissue. The tissue may also include lymphatic system and blood vessels, and has intrinsic fluid absorption and retention properties. These characteristics vary among individuals, location within the body, and over the course of an injection may cause variable degrees of resistance to the infusion of fluids at the site of injection. When the resistance of the tissue is too high or the absorption rate is too low for a given delivery flow rate from the device, the pressure may build up and reach values above the threshold where the fluid line and other components may be compromised.

### SUMMARY OF THE INVENTION

In one aspect or embodiment, a drug delivery investigation device includes a needle insertion mechanism having a cannula, a flow path having an inlet configured to be in fluid communication with a fluid source and an outlet, connection tubing extending between the outlet of the flow path and the needle insertion mechanism, with the connection tubing in fluid communication with the cannula and the flow path, a flow sensor positioned between the inlet and the outlet of the flow path, and a pressure sensor positioned between the inlet and the outlet of the flow path.

The pressure sensor may be positioned between the outlet of the flow path and the flow sensor. The flow sensor may be an inline flow sensor. The device may further include a base, where the needle insertion mechanism, the flow path, the flow sensor, and the pressure sensor are supported by the base. The device may further include at least one adhesive pad attached to the base, where at least a portion of the adhesive pad is configured to be moveable relative to the base. The at least one adhesive pad may include a connection member configured to be connected to the base and an adhesive member attached to the connection member at one or more discrete locations to allow a portion of the adhesive member to move relative to the connection member. The cannula may be configured to extend through the adhesive pad. The adhesive pad may be a first disc-shaped pad and a second disc-shaped pad spaced from the first disc-shaped pad.

The drug delivery investigation device may include a manifold, with the manifold defining the outlet of the flow path and receiving the pressure sensor. Extension tubing may extend between the manifold and the flow sensor, where the flow path is defined by the flow sensor, the extension tubing, and the manifold, and where the extension tubing and the connection tubing are formed from a low mechanical compliance material.

The needle insertion mechanism may include a guide and a needle holder, with the needle holder configured to be received within the guide and moveable relative to the guide. The guide may include a plurality of resilient arms defining an interior space configured to receive the needle holder, with the plurality of resilient arms each including a cam surface configured to engage an engagement surface of the needle holder. The needle holder may have a first position where the engagement surface is engaged with the cam surface and a second position where the engagement surface is within the interior space and spaced from the cam surface of the guide, with the plurality of resilient arms configured to deflect radially outward when the needle holder moves from the first position to the second position. The needle holder may be configured to move from the first position to the second position upon application of a predetermined axial force to the needle holder. The needle holder may include a first luer connector attached to the cannula, a second luer connecter connected to the first luer connector and in fluid communication with the connection tubing, and an actuator body configured to be manually engaged and defining the engagement surface.

The device may include at least one processor and at least one memory storage device, with the at least one processor configured to store data from the flow sensor and the pressure sensor to the at least one memory storage device. The at least one memory storage device may be configured to be connected to an external device.

The device may include at least one ancillary sensor, where the at least one ancillary sensor is at least one of an accelerometer, a photoplethysmography sensor, an ultrasonic scanner, a strain gage configured to capture skin deformation, a temperature sensor, and an impedance sensor.

In one aspect or embodiment, a method of using the drug delivery investigation device of any of the above aspects or embodiments includes: initiating streaming of data from the flow sensor and the pressure sensor to an external device; compiling and saving the data; processing the data and generating a display; and saving processed data. The method may further include: erasing data from at least one memory storage device of the drug delivery investigation device. The method may include synchronizing the output of at least two sensors. The method may include calculating an output from data obtained from at least two sensors.

In one aspect or embodiment, a method of using the drug delivery investigation device of any of the above aspects or embodiments includes: reading and writing data from the flow sensor and the pressure sensor to the at least one memory storage device of the drug delivery investigation device. The method may further include: reading and writing data from at least one ancillary sensor, where the at least one ancillary sensor is at least one of an accelerometer, a photoplethysmography sensor, an ultrasonic scanner, a strain gage configured to capture skin deformation, a temperature sensor, and an impedance sensor. The method may include synchronizing the output of at least two sensors. The method may include calculating an output from data obtained from at least two sensors.

In a further aspect or embodiment, a drug delivery investigation device includes a flow path having an inlet configured to be in fluid communication with a fluid source and an outlet, connection tubing in fluid communication with the flow path, a flow sensor positioned between the inlet and the outlet of the flow path, and a pressure sensor positioned between the inlet and the outlet of the flow path. The connection tubing may be configured to be connected to at least one of a needle insertion mechanism, a needle, and a catheter.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a schematic view of a drug delivery investigation device according to one aspect or embodiment of the present application;

FIG. 2 is a perspective view of the drug delivery investigation device of FIG. 1;

FIG. 3 is a perspective view of the drug delivery investigation device of FIG. 1, showing the device with a cover removed;

FIG. 4 is a bottom perspective view of the drug delivery investigation device of FIG. 1;

FIG. 5 is an enlarged bottom perspective view of the drug delivery investigation device of FIG. 1;

FIG. 6 is a perspective view of the drug delivery investigation device of FIG. 1, showing the device with a cover removed;

FIG. 7 is a partial side view of the drug delivery investigation device of FIG. 1;

FIG. 8 is a top view of a data logging device according to one aspect or embodiment of the present application;

FIG. 9 is a perspective view of the drug delivery investigation device of FIG. 1, showing the device connected to a power supply, fluid source, and external device;

FIG. 10 is a perspective view of the drug delivery investigation device of FIG. 1, showing a disconnected position of a needle insertion mechanism; and

FIG. 11 is a schematic view of a method of using a drug delivery investigation device according to one aspect or embodiment of the present application.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION OF THE INVENTION

Spatial or directional terms, such as “left”, “right”, “inner”, “outer”, “above”, “below”, and the like, are not to be considered as limiting as the invention can assume various alternative orientations.

All numbers used in the specification and claims are to be understood as being modified in all instances by the term “about”. By “about” is meant a range of plus or minus ten percent of the stated value. As used in the specification and the claims, the singular form of “a”, “an”, and “the” include plural referents unless the context clearly dictates otherwise. The terms “first”, “second”, and the like are not intended to refer to any particular order or chronology, but instead refer to different conditions, properties, or elements. By “at least” is meant “greater than or equal to”.

As used herein, “at least one of” is synonymous with “one or more of”. For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

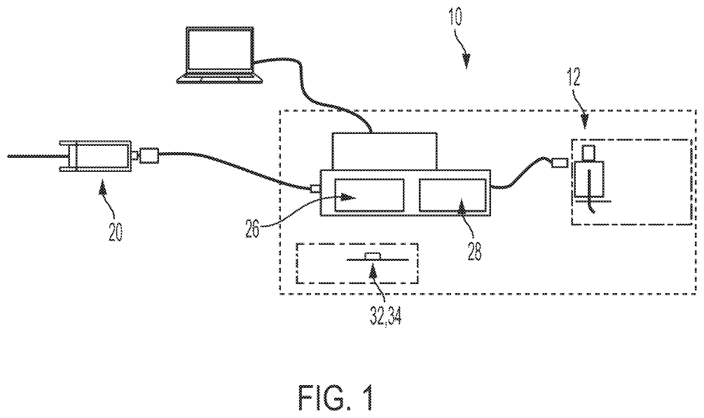

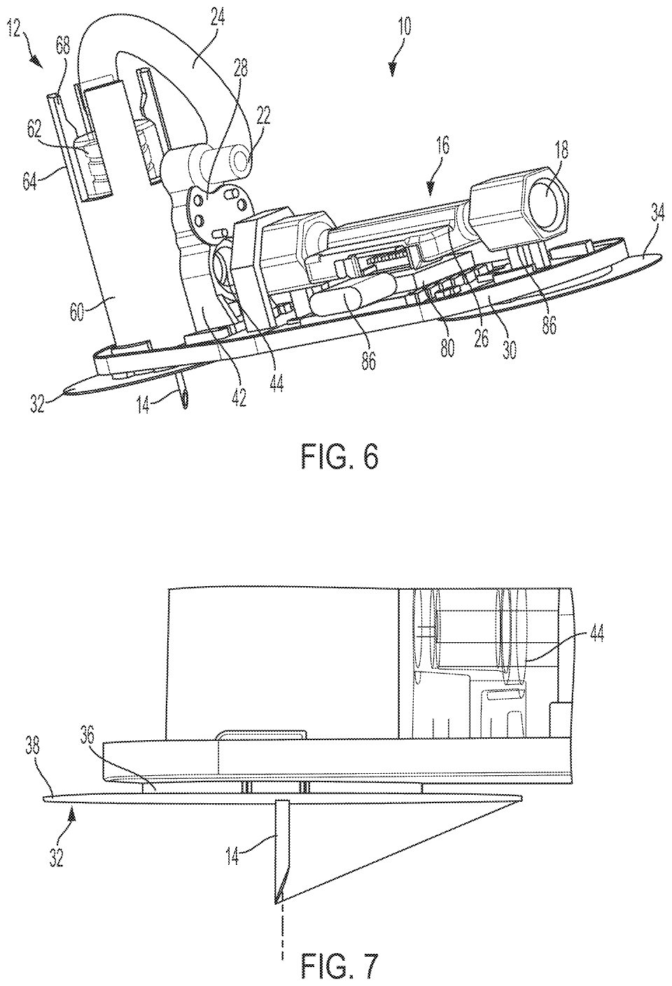

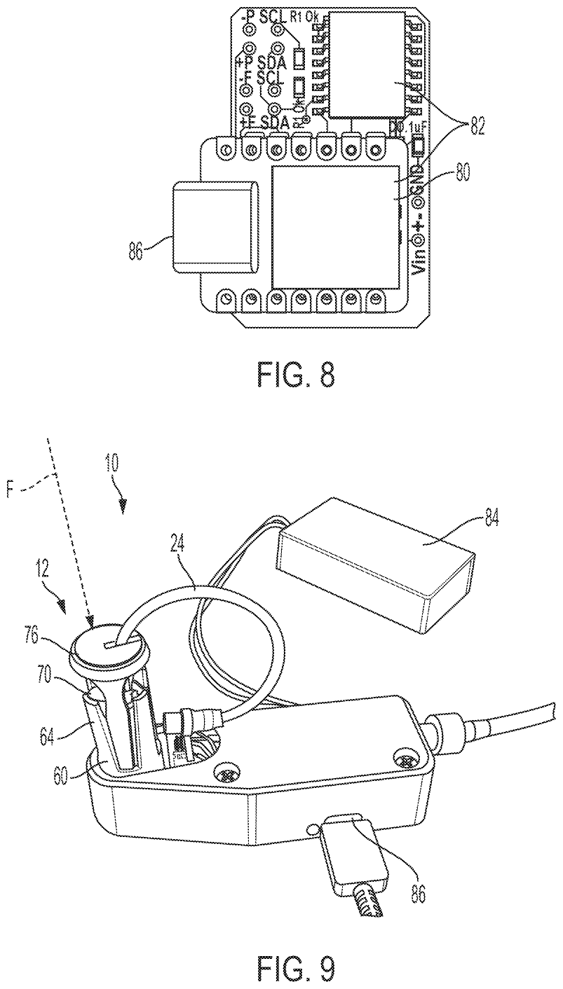

Referring to FIGS. 1-10, in one aspect or embodiment, a drug delivery investigation device 10 includes a needle insertion mechanism 12, including a cannula 14, a flow path 16 having an inlet 18 configured to be in fluid communication with a fluid source 20 and an outlet 22, connection tubing 24 extending between the outlet 22 of the flow path 16 and the needle insertion mechanism 12, a flow sensor 26 positioned between the inlet 18 and the outlet 22 of the flow path 16, and a pressure sensor 28 positioned between the inlet 18 and the outlet 22 of the flow path 16. The connection tubing 24 is in fluid communication with the cannula 14 and the flow path 16.

The drug delivery investigation device 10 is configured to measure injection parameters in-vivo and enable the characterization, assessment, and comparison of performances of injection devices. The injection parameters may be one or more of injection pressure (including peak pressure, pressure profile, average pressure, pressure decay, etc.), injection flowrate, depot formation, skin deformation (e.g., enable using an accelerometer, ultrasound, 3D scanning/reconstruction), skin bulging (wheal), etc. The drug delivery investigation device 10 is configured to enable the sensing of tissue response (e.g., tissue backpressure, fluidic resistance of tissue, tissue cracking pressure, etc.) during injection of fluid through the cannula 14. The drug delivery investigation device 10 allows for the measuring of the above parameters, which is useful in the development of a drug delivery device that use automatic or manual injection systems to deliver a dose of medication to a patient, such as through a subcutaneous injection. One example of such a drug delivery device is shown and described in U.S. Pat. No. 10,449,292 to Pizzochero et al.

During the development of such drug delivery devices, the injection parameters for a specific device and medication need to be identified and validated. Furthermore, the performances of different device or device iterations need to be quantified and objectively compared. The tissue response and in-vivo device performances are difficult to measure. The drug delivery investigation device 10 enables these difficult parameters to be measured in-vivo to obtain accurate and high quality data. As discussed in additional detail below, the drug delivery investigation device 10 is configured to minimize fluidic resistance and subsequent pressure drop between the pressure sensor 28 and the cannula 14, minimize dead volume, and minimize mechanical compliance of the flow path 16 and the connection tubing 24 between the inlet 18 and the cannula 14.

Referring again to FIGS. 1, 3, and 6, in one aspect or embodiment, the pressure sensor 28 is positioned between the outlet 22 of the flow path 16 and the flow sensor 26. The flow sensor 26 may be an inline flow sensor, although other suitable flow sensors may be utilized.

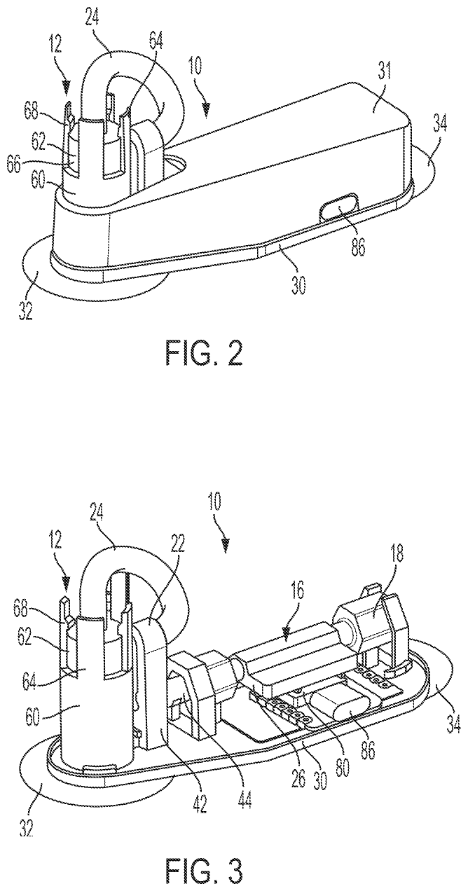

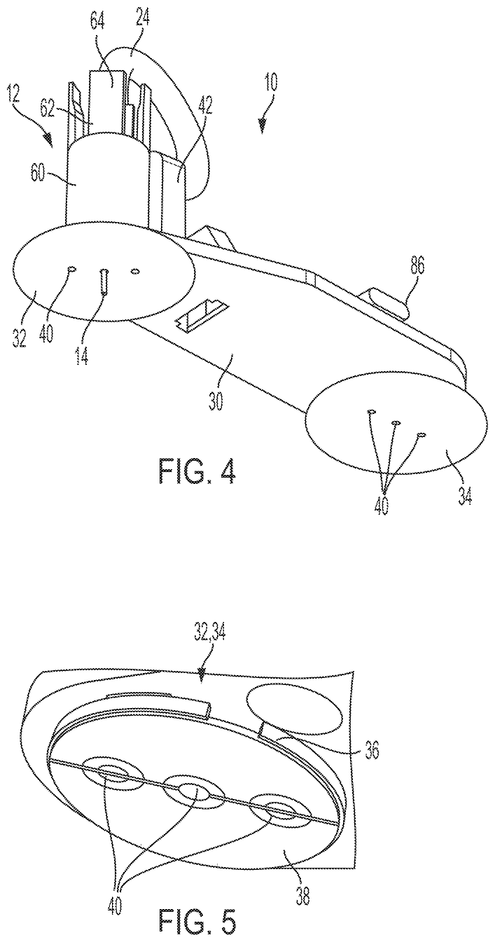

Referring to FIGS. 2-7, the drug delivery investigation device 10 includes a base 30, with the needle insertion mechanism 12, the flow path 16, the flow sensor 26, and the pressure sensor 28 supported by the base 30. A cover 31 may enclose the components of the drug delivery investigation device 10, except for the needle insertion mechanism 12. At least one adhesive pad 32, 34 is attached to the base 30, with at least a portion of the adhesive pad 32, 34 configured to be moveable relative to the base 30. The at least one adhesive pad 32, 34 may be configured to allow slight rocking or shifting of the base due to the formation of a wheal. In one aspect or embodiment, each adhesive pad 32, 34 is fixed and non-movable relative to the base 30. In one aspect or embodiment, as shown more clearly in FIGS. 4 and 5, the drug delivery investigation device 10 includes first and second disc-shaped pads 32, 34, which are spaced from each other, although other suitable shapes may be utilized. Each adhesive pad 32, 34 includes a connection member 36 configured to be connected to the base 30 and an adhesive member 38 attached to the connection member 36 at one or more discrete locations 40 to allow a portion of the adhesive member 38 to move relative to the connection member 36. In one aspect or embodiment, the adhesive member 38 is heat staked to the connection member 36 at the discrete locations only 40. The cannula 14 is configured to extend through the adhesive pads 32, 34. In one aspect or embodiment, the base 30 is configured to be a durable, reusable component while each adhesive pad 32, 34 is configured to be disposable and replaced. In one aspect or embodiment, the connection member 36 of the adhesive pads 32, 34 is connected to the base 30 via a connector arrangement (not shown). In one aspect or embodiment, the adhesive pads 32, 34 each include a removable liner (not shown) covering the adhesive member 38. The cannula 14 may be a needle cannula with a sharpened distal tip and/or a catheter.

Referring to FIGS. 1, 3, and 6, in one aspect or embodiment, the drug delivery investigation device 10 includes a manifold 42, which defines the outlet 22 of the flow path 16, with the manifold 42 receiving the pressure sensor 28. Extension tubing 44 extends between the manifold 42 and the flow sensor 26. Accordingly, in one aspect or embodiment, the flow path 16 is defined by the flow sensor 26, the extension tubing 44, and the manifold 42. In one aspect or embodiment, the extension tubing 44 and the connection tubing 24 are formed from a low mechanical compliance material. In one aspect or embodiment, the extension tubing 44 and the connection tubing 24 are formed from polytetrafluoroethylene (PTFE). In one aspect or embodiment, the connection tubing 24 has an internal diameter of 1 mm and an outer diameter of 2 mm. In one aspect or embodiment, the extension tubing 44 has an internal diameter of 1 mm and an outer diameter of 3 mm. In one aspect or embodiment, the material of the connection tubing 24 and the extension tubing 44 has a Young's modulus of 400,000 kPa.

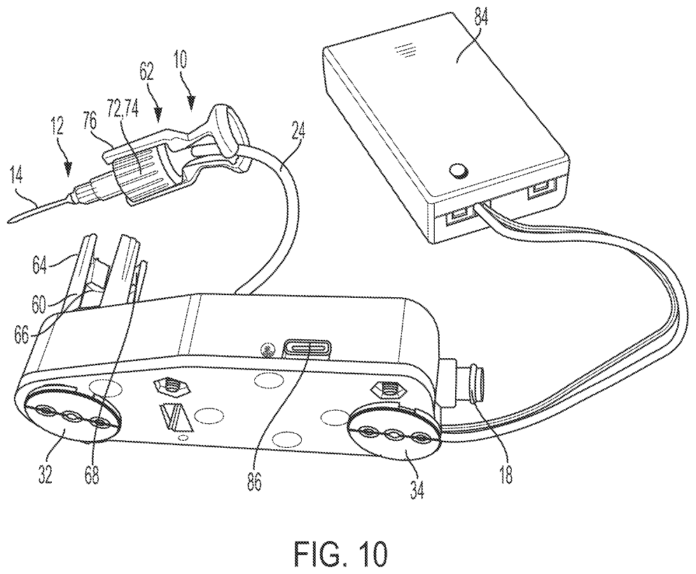

Referring to FIGS. 2-4, 6, 9, and 10, the needle insertion mechanism 12 includes a guide 60 and a needle holder 62, with the needle holder 62 configured to be received within the guide 60 and moveable relative to the guide 60. The guide 60 includes a plurality of resilient arms 64 defining an interior space 66 configured to receive the needle holder 62. In one aspect or embodiment, as shown in FIGS. 2-4, the guide 60 includes four resilient arms 64. The plurality of resilient arms 64 each include a cam surface 68 configured to engage an engagement surface 70 of the needle holder 62. The needle holder 62 has a first position where the engagement surface 70 is engaged with the cam surface 68 and a second position where the engagement surface 70 is within the interior space 66 and spaced from the cam surface 68 of the guide 60. The plurality of resilient arms 64 are each configured to deflect radially outward when the needle holder 62 moves from the first position to the second position. The needle holder 62 is configured to move from the first position to the second position upon application of a predetermined axial force F to the needle holder 62. The needle holder 62 and the guide 60 are configured to provide a semi-ballistic insertion of the cannula 14. In one aspect or embodiment, the needle holder 62 includes a first luer connector 72 attached to the cannula 14, a second luer connecter 74 connected to the first luer connector 72 and in fluid communication with the connection tubing 24, and an actuator body 76 configured to be manually engaged and defining the engagement surface 70. As shown more clearly in FIG. 10, the actuator body 76 may receive the first luer connector 72 and the second luer connector 74. The actuator body 76 can be grasped and inserted into the guide 60. Upon application of the predetermined axial force F, which can be applied through the actuator body 76, the needle holder 62 and actuator body 76 are moved axially within the guide 60, with the engagement surface 70 of the actuator body 76 engaging the cam surfaces 68 of the plurality of resilient arms 64 to bias the plurality of resilient arms 64 radially outward. When the engagement surface 70 of the actuator body 76 axially moves past the cam surfaces 68, the needle holder 62 and the actuator body 76 are quickly unrestrained or released by the guide 60 which provides for a semi-ballistic insertion of the cannula 14. In one aspect or embodiment, a depth of insertion of the cannula 14 may be adjustable.

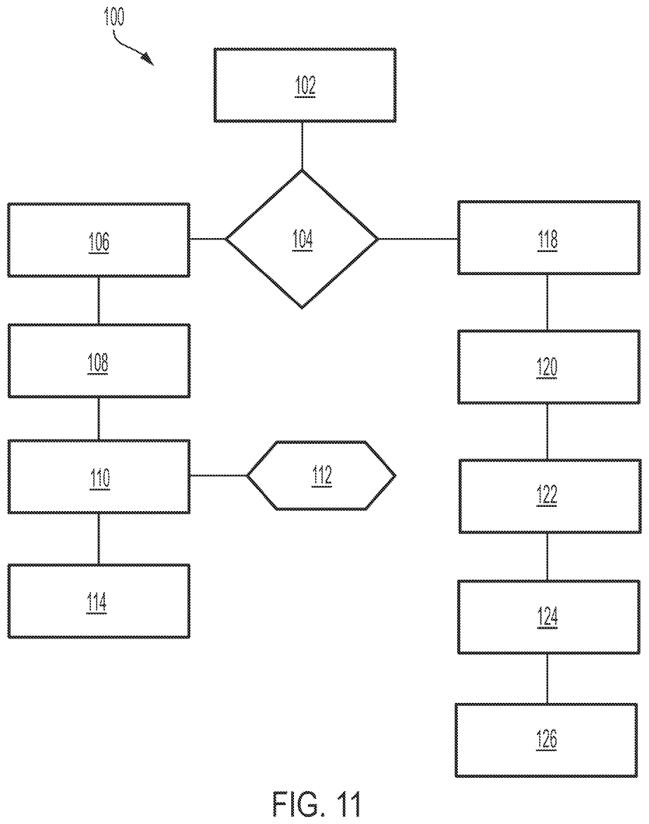

Referring to FIGS. 1, 3, and 8-11, the drug delivery investigation device 10 includes at least one processor 80 and at least one memory storage device 82. The processor 80 is configured to store data from the flow sensor 26 and the pressure sensor 28 to the memory storage device 82. The memory storage device 82 is configured to be connected to an external device, such as a computer. In one aspect or embodiment, the processor 80 is an Arduino microcontroller with RAM and ROM memory. The memory storage device 82 may also include flash memory for storing data. The drug delivery investigation device 10 also includes an external power source 84, such as a battery pack, although an internal power source or other power source may utilized. The drug delivery investigation device 10 also includes a port 86, such as a USB port, to connect the drug delivery investigation device 10 to an external device, such as a computer, for reading data, streaming data, resetting the device, and/or controlling the device. In one aspect or embodiment, the drug delivery investigation device 10 is configured to wirelessly transmit data and/or be controlled wirelessly.

Referring to FIG. 12, in one aspect or embodiment, a method 100 of using the drug delivery investigation device 10 includes initiating the device 102 and selecting a live streaming of data or local saving of data 104. If live streaming of data is selected, the method 100 further includes: initiating streaming of data from the flow sensor and the pressure sensor to an external device 106; compiling and saving the data 108; processing the data 110 and generating a display 112; and saving the processed data 114. The method 100 may also include erasing data from the memory storage device 82 of the drug delivery investigation device 10. If local saving of data is selected, the method 100 further includes: reading and writing data 118 from the flow sensor 26 and the pressure sensor 28 to the memory storage device 82 of the drug delivery investigation device 10. The data may be saved to the flash memory. In one aspect or embodiment, the local saving of data includes: reading and writing data to a timestamp every 40 ms 118; logging the data to a first buffer of 90 timestamps 120; writing the buffer to a first page of memory 122; clearing the buffer and logging a second buffer of 90 timestamps 124; and saving the second buffer to a second page of memory 126. Each timestamp may include pressure, volume flow rate, temperature, and time.

In one aspect or embodiment, the method 100 further includes reading and writing data from at least one ancillary sensor, where the at least one ancillary sensor is at least one of an accelerometer, a photoplethysmography sensor, an ultrasonic scanner, a strain gage configured to capture skin deformation, a temperature sensor, and an impedance sensor. In one aspect or embodiment, the method 100 includes synchronizing the output of at least two sensors. In one aspect or embodiment, the method 100 includes calculating an output from data obtained from at least two sensors.

In one aspect or embodiment, the drug delivery investigation device 10 is configured to be primed to remove air from the flow path 16, the flow sensor 26, the pressure sensor 28, the connection tubing 24, and the extension tubing 44. The drug delivery investigation device 10 may include a sensor to enable the measurement of the depot formation and location, such as through the use of photoplethysmography, impedance tomography, ultrasonic scanner probes, thermal imaging, contact or contactless thermal sensors disposed to measure the temperature at a skin surface, strain gage capturing skin deformation, or any combination thereof.

In one aspect or embodiment, the fluid source 20, which is connectable to the inlet 18 of the flow path 16, may be a syringe pump, a peristaltic pump, a manually activated syringe, or other suitable arrangement.

In one aspect or embodiment, the flow path 16 of the drug delivery investigation device 10 is configured with channel dimension to constitute a maximum 80% of the fluidic resistance of the tissue. In one aspect or embodiment, the flow path 16 of the drug delivery investigation device 10 is configured with channel dimension to constitute a maximum 90% of the fluidic resistance of the tissue.

In one aspect or embodiment, the materials of the drug delivery investigation device 10 are selected to constitute maximum 80% of the mechanical compliance of the drug delivery investigation device 10.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A drug delivery investigation device comprising:
a needle insertion mechanism comprising a cannula;
a flow path comprising an inlet configured to be in fluid communication with a fluid source and an outlet;
connection tubing extending between the outlet of the flow path and the needle insertion mechanism, the connection tubing in fluid communication with the cannula and the flow path;
a flow sensor positioned between the inlet and the outlet of the flow path; and
a pressure sensor positioned between the inlet and the outlet of the flow path.

2. The drug delivery investigation device of claim 1, wherein the pressure sensor is positioned between the outlet of the flow path and the flow sensor.

3. The drug delivery investigation device of claim 1, wherein the flow sensor comprises an inline flow sensor.

4. The drug delivery investigation device of claim 1, further comprising a base, wherein the needle insertion mechanism, the flow path, the flow sensor, and the pressure sensor are supported by the base.

5. The drug delivery investigation device of claim 4, further comprising at least one adhesive pad attached to the base.

6. The drug delivery investigation device of claim 5, wherein at least a portion of the adhesive pad is configured to be moveable relative to the base, and wherein the at least one adhesive pad comprises a connection member configured to be connected to the base and an adhesive member attached to the connection member at one or more discrete locations to allow a portion of the adhesive member to move relative to the connection member.

7. The drug delivery investigation device of claim 5, wherein the cannula is configured to extend through the adhesive pad.

8. The drug delivery investigation device of claim 5, wherein the adhesive pad comprises a first disc-shaped pad and a second disc-shaped pad spaced from the first disc-shaped pad.

9. The drug delivery investigation device of claim 1, further comprising a manifold, the manifold defining the outlet of the flow path, the manifold receiving the pressure sensor.

10. The drug delivery investigation device of claim 9, wherein extension tubing extends between the manifold and the flow sensor, wherein the flow path is defined by the flow sensor, the extension tubing, and the manifold, and wherein the extension tubing and the connection tubing are formed from a low mechanical compliance material.

11. The drug delivery investigation device of claim 1, wherein the needle insertion mechanism comprises a guide and a needle holder, the needle holder configured to be received within the guide and moveable relative to the guide.

12. The drug delivery investigation device of claim 11, wherein the guide comprises a plurality of resilient arms defining an interior space configured to receive the needle holder, the plurality of resilient arms each comprising a cam surface configured to engage an engagement surface of the needle holder, and wherein the needle holder has a first position where the engagement surface is engaged with the cam surface and a second position where the engagement surface is within the interior space and spaced from the cam surface of the guide, the plurality of resilient arms configured to deflect radially outward when the needle holder moves from the first position to the second position.

13. The drug delivery investigation device of claim 12, wherein the needle holder is configured to move from the first position to the second position upon application of a predetermined axial force to the needle holder.

14. The drug delivery investigation device of claim 13, wherein the needle holder comprises a first luer connector attached to the cannula, a second luer connecter connected to the first luer connector and in fluid communication with the connection tubing, and an actuator body configured to be manually engaged and defining the engagement surface.

15. The drug delivery investigation device of claim 1, further comprising at least one processor and at least one memory storage device, the at least one processor configured to store data from the flow sensor and the pressure sensor to the at least one memory storage device.

16. The drug delivery investigation device of claim 15, wherein the at least one memory storage device is configured to be connected to an external device.

17. The drug delivery investigation device of claim 1, further comprising at least one ancillary sensor, the at least one ancillary sensor comprising at least one of an accelerometer, a photoplethysmography sensor, an ultrasonic scanner, a strain gage configured to capture skin deformation, a temperature sensor, and an impedance sensor.

18.-25. (canceled)
